(19) United States
(12) Patent Application Publication
(10) Pub. No.: US 2021/0244672 A1
(43) Pub. Date: Aug. 12, 2021

(54) Ingestible Product and a Method of Using the Same

(71) Applicant: Tremeau Pharmaceuticals, Inc., Cambridge, MA (US)

(72) Inventor: Bradford C. Sippy, Acton, MA (US)

(21) Appl. No.: 17/242,759

(22) Filed: Apr. 28, 2021

Related U.S. Application Data
(63) Continuation of application No. 16/138,564, filed on Sep. 21, 2018, now abandoned.
(60) Provisional application No. 62/561,595, filed on Sep. 21, 2017.

(51) Int. Cl. A61K 9/20; A61K 31/454; G16H 20/10; G16H 10/40; A61B 5/07; A61B 5/00; A61K 31/444; G16H 40/67; A61K 31/341; A61K 31/40

(52) U.S. Cl. CPC A61K 9/2072; A61K 31/454; G16H 20/10; G16H 10/40; A61B 5/073; A61K 31/40; A61K 31/444; G16H 40/67; A61K 31/341; A61B 5/7275; A61B 5/4839

(57) ABSTRACT

An ingestible product is configured to be swallowed by a patient and includes: a drug portion that requires a Risk Evaluation and Mitigation Strategy (REMS) plan; and a wireless sensor portion configured to transmit sensor data concerning the drug portion to a remote device.

### RELATED APPLICATION(S)

This application claims the benefit of U.S. Provisional Application No. 62/561,595, filed on 21 Sep. 2017, the entire contents of which are incorporated herein by reference.

### TECHNICAL FIELD

This disclosure relates to ingestible products and, more particularly, to ingestible products that include a wireless sensor portion.

### BACKGROUND

The field of ingestible sensor technology has made significant advances with respect to e.g., design variety, communication methodologies, and measurement goals. For example, ingestible sensors have been well developed to minimize their size, improve their information transfer capabilities, and establish a physiological-based source of power. These advances have enabled the utilization of ingestible sensor technology to progress into human clinical applications, with the most direct application being to utilize the data generated via the concomitant administration of an ingestible sensor with a pharmaceutical product to improve the tracking of the timing of administration of a pharmaceutical product.

### SUMMARY OF DISCLOSURE

In one implementation, an ingestible product is configured to be swallowed by a patient and includes: a drug portion that requires a Risk Evaluation and Mitigation Strategy (REMS) plan; and a wireless sensor portion configured to transmit sensor data concerning the drug portion to a remote device.

One or more of the following features may be included. The drug portion may be chosen from the group consisting of: a COX-2 Selective Nonsteroidal Anti-inflammatory Drug (NSAID); and a D2 receptor antagonist. The wireless sensor portion may be configured to wirelessly transmit the sensor data via a wireless communication protocol. The wireless communication protocol may be configured to couple the wireless sensor portion to the remote device. The sensor data may include: identification information concerning the drug portion; and dosage information concerning the drug portion. The remote device may be chosen from the group consisting of: a smart phone; a smart watch; a wearable patch; a dedicated external device; and a remote computing system.

In another implementation, a computer program product resides on a computer readable medium and has a plurality of instructions stored on it. When executed by a processor, the instructions cause the processor to perform operations including: receiving sensor data from a wireless sensor portion of an ingestible product, wherein the sensor data concerns a drug portion of the ingestible product that requires a Risk Evaluation and Mitigation Strategy (REMS) plan; generating drug portion information; and providing the drug portion information to a third party. The drug portion information is based, at least in part, upon the sensor data.

One or more of the following features may be included. Supplemental data may be received from a user. The drug portion information may be based, at least in part, upon the supplemental data. The supplemental data may be chosen from the group consisting of: reaction information; condition information; status information; and biographical information. The ingestible product may be chosen from the group consisting of a: a pill; a capsule; and a gelcap. The drug portion may be chosen from the group consisting of: a COX-2 Selective Nonsteroidal Anti-inflammatory Drug (NSAID); and a D2 receptor antagonist. The third party may be chosen from the group consisting of: a patient; a user; a medical professional; a regulatory authority; a pharmaceutical professional; and an insurance professional. The sensor data may include: identification information concerning the drug portion; and dosage information concerning the drug portion.

In another implementation, a computing system includes a processor and memory is configured to perform operations including: receiving sensor data from a wireless sensor portion of an ingestible product, wherein the sensor data concerns a drug portion of the ingestible product that requires a Risk Evaluation and Mitigation Strategy (REMS) plan; generating drug portion information; and providing the drug portion information to a third party. The drug portion information is based, at least in part, upon the sensor data.

One or more of the following features may be included. Supplemental data may be received from a user. The drug portion information may be based, at least in part, upon the supplemental data. The supplemental data may be chosen from the group consisting of: reaction information; condition information; status information; and biographical information. The ingestible product may be chosen from the group consisting of a: a pill; a capsule; and a gelcap. The drug portion may be chosen from the group consisting of: a COX-2 Selective Nonsteroidal Anti-inflammatory Drug (NSAID); and a D2 receptor antagonist. The third party may be chosen from the group consisting of: a patient; a user; a medical professional; a regulatory authority; a pharmaceutical professional; and an insurance professional. The sensor data may include: identification information concerning the drug portion; and dosage information concerning the drug portion.

The details of one or more implementations are set forth in the accompanying drawings and the description below. Other features and advantages will become apparent from the description, the drawings, and the claims.

### BRIEF DESCRIPTION OF THE DRAWINGS

FIG. 1 is a diagrammatic view of an ingestible product wirelessly coupled to a remote device; and

FIG. 2 is a flow chart of one implementation of a monitoring process configured to process data received from the ingestible product of FIG. 1.

Like reference symbols in the various drawings indicate like elements.

### DETAILED DESCRIPTION OF THE PREFERRED EMBODIMENTS

### System Overview:

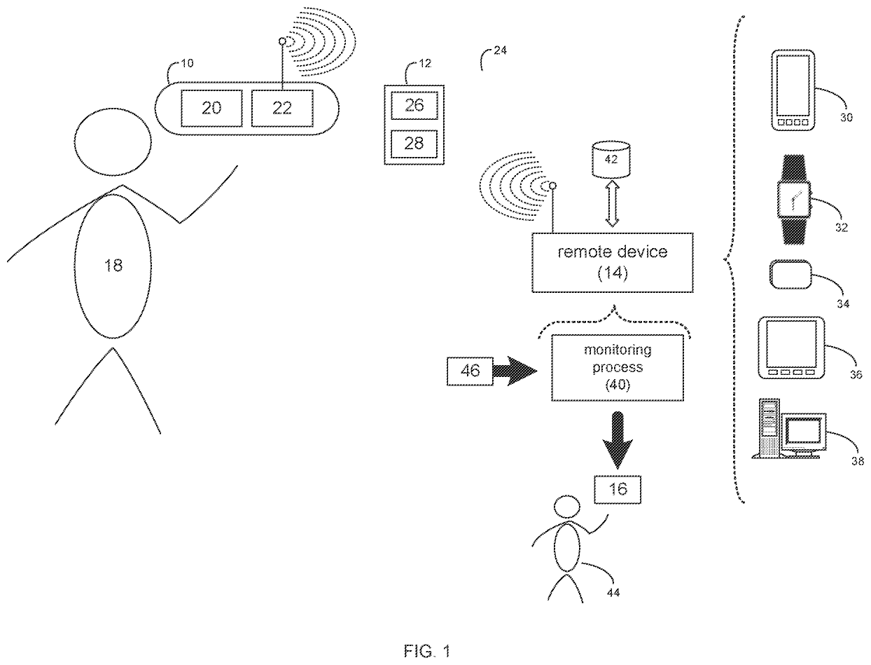

Referring to FIG. 1, there is shown ingestible product 10. As will be discussed below in greater detail, ingestible product 10 may be configured to transmit sensor data 12 to remote device 14, wherein sensor data 12 may be utilized to generate drug portion information 16.

Ingestible product 10, which may be configured to be swallowed by a patient (e.g., patient 18), may include: drug portion 20 that requires a Risk Evaluation and Mitigation Strategy (REMS) plan; and wireless sensor portion 22 configured to transmit sensor data 12 concerning drug portion 20 to remote device 14. Examples of ingestible product 10 may include but are not limited to: a pill; a capsule; and a gelcap.

As is known in the art, Risk Evaluation and Mitigation Strategies is a program of the US Food and Drug Administration for the monitoring of medications with a high potential for serious adverse effects. The REMS program was formalized in 2007. If the FDA determines as part of the drug approvals process that a REMS is necessary, the drug company develops and maintains the individual program. While REMS applies only to specific prescription drugs, it can apply to brand name or generic drugs. REMS for generic drugs may be created in collaboration with the manufacture of the brand name drug. Further, the FDA may remove the REMS requirement if it is found to not improve patient safety.

The REMS program developed out of previous systems dating back to the 1980s for monitoring the use of a small number of high-risk drugs such as the Accutane, which causes serious birth defects, Clozaril, which can cause agranulocytosis, and Thalidomide, which is used to treat leprosy but causes serious birth defects. The 2007 Food and Drug Administration Amendments Act created § 505-1 of the Food, Drug, and Cosmetic Act which allowed for the creation of the REMS program for applying individual monitoring restrictions to medications. Some of the provisions required by the REMS program are training and certification of physicians allowed to prescribe the drug, requiring that the drug be administered in a hospital setting, requiring pharmacies to verify the status of patients receiving REMS drugs, requiring lab testing of patients to ensure that health status is satisfactory, or requiring that patients be entered into a registry.

Accordingly, a REMS program is typically a strategic safety program designed to meet specific goals and objectives in minimizing known risks of a product identified through the product development process or based on other information. Typically, a suitable REMS program targets one or more safety-related health outcomes or goals and uses one or more tools to achieve those goals. Over time, the effectiveness of the tools is evaluated and the benefit-risk balance for the product is reassessed. During patient follow-up, appropriate adjustments are then made to the risk minimization tools to further improve the benefit-risk balance. A REMS for an NDA or BLA may contain any of the following elements:
Medication Guide or Patient Package Insert;
Communication Plan;
Elements To Assure Safe Use (ETASU); and/or
Implementation System.

The ETASU requirements are intended to reduce a specific serious risk listed in the labeling of the drug. Depending on the risks associated with a drug, an ETASU may require any of the following:
Prescribers have specific training/experience or special certifications;
Pharmacies, practitioners or healthcare settings that dispense the drug be specially certified;
Drug be dispensed only in certain healthcare settings (e.g., infusion settings, hospitals);
Drug be dispensed with evidence of safe-use conditions such as laboratory test results;
Each patient using the drug be subject to monitoring; and/or
Each patient using the drug be enrolled in a registry.

However, in order for the ETASU to be effective, it must not unduly burden patients, healthcare professionals or the healthcare system. The following provisions help ensure REMS are as efficient as possible:
ETASU requirements must be commensurate with the specific serious risk listed in the drug's labeling;
Cannot be unduly burdensome on patient access to the drug, especially those who have serious or life-threatening diseases and/or difficulty accessing healthcare; and
To the extent practicable, ETASU must conform with other components for other drugs with similar serious risks and be designed to be compatible with established distribution, procurement, and dispensing systems for drugs.

An example of drug portion 20 may include but is not limited to a COX-2 Selective Nonsteroidal Anti-inflammatory Drug (NSAID). As is known in the art, an NSAID is generally a drug class that reduces pain, decreases fever, prevents blood clots and, in higher doses, decreases inflammation. Side effects depend on the specific drug, but largely include an increased risk of gastrointestinal ulcers and bleeds, heart attack and kidney disease. The term nonsteroidal distinguishes these drugs from steroids, which while having a similar eicosanoid-depressing, anti-inflammatory action, have a broad range of other effects. First used in 1960, the term served to distance these medications from steroids. NSAIDs work by inhibiting the activity of cyclooxygenase enzymes (COX-1 and/or COX-2). In cells, these enzymes are involved in the synthesis of key biological mediators, namely prostaglandins (which are involved in inflammation) and thromboxanes (which are involved in blood clotting). COX-2 Selective NSAIDs are designed specifically to inhibit only the COX-2 enzyme, and thus share the anti-inflammatory benefits of NSAIDs with a differentiated safety profile due to the absence of COX-1 inhibition.

Specific examples of such COX-2 Selective NSAIDs may include but are not limited to:
ROFECOXIB:
   Rofecoxib is a COX-2 selective non-steroidal anti-inflammatory drug (NSAID) that has been well-characterized in pre-marketing nonclinical and clinical studies.
   Rofecoxib was originally available in 12.5 mg, 25 mg, 50 mg. 50 mg was only approved for acute use, though some patients used 50 mg chronically off-label. 12.5 mg and 25 mg both comparable in efficacy to maximum dose traditional NSAIDs.
   Rofecoxib, unlike non-selective NSAIDs, does not affect platelet aggregation.
   Rofecoxib was the only product approved in the U.S. in the COX-2 class with demonstrated GI safety advantage vs. a non-selective NSAID.
   Rofecoxib was withdrawn from the market in 2004 due to evidence of a CV safety issue and has never been returned to the market.
   Subsequent data indicates that the CV safety issue was not unique to rofecoxib, but a dose dependent class effect of all NSAIDs.
   12.5 mg and 25 mg of rofecoxib appear to have similar CV risk as other NSAIDs, 50 mg has higher risk than other NSAIDs.
   FDA and other regulatory authorities modified the safety information for COX-2 selective and non-selective NSAIDs, emphasizing using the lowest effective dose for shortest duration.
   Rofecoxib has one or more unique benefits (GI safety, lack of anti-platelet effect) and no unique risks versus other NSAIDs when used at lower doses and/or for shorter durations.
   An ingestible product (e.g., ingestible product 10) that includes wireless sensor portion 22 and drug portion 20 that includes rofecoxib as an active ingredient may help support safety programs designed to: ensure higher doses are not utilized; track potential adverse events; and/or match patient pain feedback with the dose utilized, all with the potential of supporting the use of the lowest effective dose for the shortest duration or other safety goals.
ETORICOXIB:
   Etoricoxib is a COX-2 selective non-steroidal anti-inflammatory drug (NSAID) that has been well-characterized in pre-marketing nonclinical and clinical studies.
   Available in 30 mg, 60 mg, 90 mg, and 120 mg doses. The lower doses, 30 mg and 60 mg, are both comparable in efficacy to maximum dose traditional NSAIDs.
   In chronic use, significantly higher hypertension related adverse events with 90 mg vs 60 mg or etoricoxib.
   Etoricoxib, unlike non-selective NSAIDs, does not affect platelet aggregation.
   Etoricoxib demonstrated a significant difference in GI ulcers vs diclofenac, a widely prescribed non-selective NSAID, and no difference in CV risk in the large CV/GI outcome study MEDAL.
   Etoricoxib is available in most countries, but notably never approved in the United States, Japan, or Canada.
   FDA and other regulatory authorities modified the safety information for COX-2 selective and non-selective NSAIDs, including etoricoxib in countries where it is available, emphasizing using the lowest effective dose for shortest duration.
   30 mg and 60 mg of etoricoxib appear to have similar CV risk as other NSAIDs.
   Etoricoxib has one or more unique benefits (GI safety, lack of anti-platelet effect) and no unique risks versus other NSAIDs when used at lower doses and/or for shorter durations.
   An ingestible product (e.g., ingestible product 10) that includes wireless sensor portion 22 and drug portion 20 that includes etoricoxib as an active ingredient would help support safety programs designed to ensure higher doses are not utilized, or track potential adverse events, or match patient pain feedback with the dose utilized, all with the potential of supporting the use of the lowest effective dose for the shortest duration or other safety goals.

Another example of drug portion 20 may include but is not limited to a D2 receptor antagonist. As is known in the art, a receptor antagonist is general a type of receptor ligand or drug that blocks or dampens a biological response by binding to and blocking a receptor rather than activating it like an agonist. They are sometimes called blockers; examples of which may include but are not limited to alpha blockers, beta blockers, and calcium channel blockers. In pharmacology, antagonists have affinity but no efficacy for their cognate receptors, and binding will disrupt the interaction and inhibit the function of an agonist or inverse agonist at receptors. Antagonists mediate their effects by binding to the active site or to the allosteric site on a receptor, or they may interact at unique binding sites not normally involved in the biological regulation of the receptor's activity. Antagonist activity may be reversible or irreversible depending on the longevity of the antagonist-receptor complex, which, in turn, depends on the nature of antagonist-receptor binding. The majority of drug antagonists achieve their potency by competing with endogenous ligands or substrates at structurally defined binding sites on receptors. D2 receptor antagonists are a specific type of receptor antagonist useful in treating several conditions, including schizophrenia, bipolar disorder, nausea and vomiting, but may also produce side effects associated with D2 antagonism.

Specific examples of such D2 receptor antagonists may include but are not limited to:
AMISULPRIDE:
   Amisulpride is an atypical antipsychotic, a potent D2 and D3 receptor antagonist, and is used to treat psychosis in schizophrenia, episodes of mania in bipolar disorder and dysthymia.
   Amisulpride is considered superior to some other atypical antipsychotics in the treatment of psychosis.
   Amisulpride is associated with several dose related side effects, including extrapyramidal side effects, QT prolongation, tardive dyskinesia, and torsades de pointes.
   Prodromes of these side effects are often asymptomatic but could be detected with use of an ingestible sensor and associated external sensor.
   An ingestible product (e.g., ingestible product 10) that includes wireless sensor portion 22 and drug portion 20 that includes amilsulpride as an active ingredient would help support the safe and effective use of amilsulpride by monitoring surrogates or prodromes of potential significant sides effects and to match dose, timing of dose, etc. to allow for dose modification or product discontinuation prior to development of overt side effects.
DOMPERIDONE:
   Domperidone is a peripherally selective dopamine D2 receptor antagonist and is used as an antiemetic, gastroprokinetic agent, and galactagogue.
   Domperidone is associated with several potential side effects, in particular dose related QT prolongation and cardiac arrest.
   Prodromes of these side effects are often asymptomatic but could be detected with use of an ingestible sensor and associated external sensor.
   An ingestible product (e.g., ingestible product 10) that includes wireless sensor portion 22 and drug portion 20 that includes domperidone as an active ingredient would help support the safe and effective use of domperidone by monitoring surrogates or prodromes of potential significant sides effects and to matched dose, timing of dose, etc. to allow for dose modification or product discontinuation prior to development of overt side effects.

Wireless sensor portion 22 may be configured to wirelessly transmit sensor data via a wireless communication protocol. For example, wireless sensor portion 22 may be configured an IEEE 802.11a, 802.11b, 802.11g, 802.11n, Wi-Fi, and/or Bluetooth device that is capable of establishing wireless communication channel 24 to couple wireless sensor portion 22 and remote device 14.

As is known in the art, IEEE 802.11x specifications may use Ethernet protocol and carrier sense multiple access with collision avoidance (i.e., CSMA/CA) for path sharing. The various 802.11x specifications may use phase-shift keying (i.e., PSK) modulation or complementary code keying (i.e., CCK) modulation, for example. As is known in the art, Bluetooth is a telecommunications industry specification that allows e.g., mobile phones, computers, and personal digital assistants to be interconnected using a short-range wireless connection. Alternatively still, wireless sensor portion 22 may be configured to work with other communication protocols/configurations/methodologies.

Sensor data 12 may include: identification information 26 concerning drug portion 20; and dosage information 28 concerning drug portion 20. Examples of remote device 14 may include but are not limited to: smart phone 30; smart watch 32; wearable patch 34; dedicated external device 36; and remote computing system 38. Remote device 14 may execute an operating system, examples of which may include but are not limited to Microsoft Windows™, Android™, WebOS™, iOS™, Redhat Linux™, or a custom operating system. As will be discussed below in greater detail, remote device 14 may be configured to execute monitoring process 40.

Smart Phone:

A traditional smart phone, such as an iPhone™ or an Android phone, may be configured to work with ingestible product 10 generally (and wireless sensor portion 22 specifically). For example, a dedicated application may be installed on smart phone 30 that may be configured to receive sensor data 12 from wireless sensor portion 22 of ingestible product 10 and, passively or actively, gather supplemental data including but not limited to: reaction information; condition information; status information; and biographical information.

Smart Watch:

A traditional smart watch, such as an Apple Watch™ or a FitBit™, may be configured to work with ingestible product 10 generally (and wireless sensor portion 22 specifically). For example, a dedicated application may be installed on smart watch 32 that may be configured to receive sensor data 12 from wireless sensor portion 22 of ingestible product 10 and, passively or actively, gather supplemental data including but not limited to: reaction information; condition information; status information; and biographical information.

Wearable Patch:

A wearable patch, such as an adhesive-backed transceiver, may be configured to work with ingestible product 10 generally (and wireless sensor portion 22 specifically). For example, wearable patch 34 may be an intermediary receiver & transmitter, wherein wearable patch 34 may be configured to receive sensor data 12 from wireless sensor portion 22 of ingestible product 10 and may be configured to retransmit sensor data 12 to a remote device (such as smart phone 30; smart watch 32; dedicated external device 36; and/or remote computing system 38).

Dedicated External Device:

A dedicated external device may be configured to work with ingestible product 10 generally (and wireless sensor portion 22 specifically). For example, dedicated external device 36 may be configured as an IEEE 802.11a, 802.11b, 802.11g, 802.11n, Wi-Fi, and/or Bluetooth device that may receive sensor data 12 from wireless sensor portion 22 of ingestible product 10. Dedicated external device 36 may also be configured to transmit (via wireless transmission or wired transmission) sensor data 12 to a remote device (such as smart phone 30; smart watch 32; and remote computing system 38).

Remote Computing System:

A remote computing system, such as a traditional Windows™ or Mac™ computer, may be configured to work with ingestible product 10 generally (and wireless sensor portion 22 specifically). For example, remote computing system 38 may be configured to receive (either directly or indirectly) sensor data 12 from wireless sensor portion 22 of ingestible product 10.

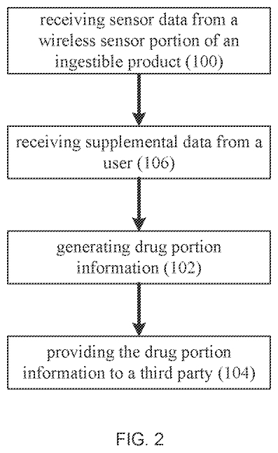

Referring also to FIG. 2, monitoring process 40 may be configured to process sensor data 12 received from wireless sensor portion 22 of ingestible product 10. Examples of monitoring process 40 may include but are not limited to a web browser or a specialized application (e.g., an application running on e.g., the Android platform, the iOS platform, the Windows platform or the Mac platform). The instruction sets and subroutines of monitoring process 40, which may be stored on storage device 42 coupled to remote device 14, may be executed by one or more processors (not shown) and one or more memory architectures (not shown) incorporated into remote device 14. Examples of storage device 42 may include but are not limited to: a hard disk drive; a random access memory (RAM); a read-only memory (ROM), and all forms of flash memory storage devices.

Monitoring process 40 may be configured to receive 100 sensor data 12 from wireless sensor portion 22 of ingestible product 10. Sensor data 12 may concern drug portion 20 of ingestible product 10, wherein drug portion 20 may require a Risk Evaluation and Mitigation Strategy (REMS) plan. As discussed above, examples of drug portion 20 may include but are not limited to a COX-2 Selective Nonsteroidal Anti-inflammatory Drug (NSAID) and a D2 receptor antagonist.

As discussed above, examples of ingestible product 10 may include but are not limited to: a pill; a capsule; and a gelcap. Once ingested by e.g., patient 18, ingestible product 10 may dissolve within the stomach of the ingester (e.g., patient 18) or naturally eliminated through the ingester's GI tract. Therefore and after ingestion, wireless sensor portion 22 may be exposed to various acids within the stomach of the ingester (e.g., patient 18). Accordingly, wireless sensor portion 22 may be configured to include a metallic anode (not shown) and a metallic cathode (not shown) that, when exposed to the various acids within the stomach of the ingester (e.g., patient 18), may generate electrical energy that may power wireless sensor portion 22.

In such a configuration, wireless sensor portion 22 may be unpowered prior to being ingested by the ingester (e.g., patient 18). However and shortly after being ingested by patient 18, wireless sensor portion 22 may be exposed to the acids within the stomach of the ingester (e.g., patient 18), thus powering wireless sensor portion 22 and enabling the transmission of sensor data 12 to remote device 14. As discussed above, examples of sensor data 12 may include but are not limited to: identification information 26 concerning drug portion 20; and dosage information 28 concerning drug portion 20.

Accordingly and shortly after the ingestation of ingestible product 10, wireless sensor portion 22 may be energized and sensor data 12 may be transmitted to remote device 14, wherein sensor data 12 may define the specifics of drug portion 20. For example, sensor data 12 may include identification information 26 that defines the particular drug as rofecoxib and dosage information 28 that defines the particular dose as 25 milligram.

Upon receiving 100 sensor data 12 concerning (in this example) a 25 milligram dose of rofecoxib being taken by patient 18, monitoring process 40 may assign a time stamp to sensor data 12, thus defining a timeline for the ingestation of drug portion 20. Accordingly, if the ingester (e.g., patient 18) is prescribed a bottle of ten pills (i.e., ten of ingestible product 10) that patient 18 is supposed to take once per day for up to fifteen days as needed for pain, monitoring process 40 may track utilization of the drug product by monitoring for the receipt 100 of sensor data for each of the ten ingestible products and associating a time stamp with each of these pieces of sensor data. Therefore and if the ingester (e.g., patient 18) follows the defined dosage schedule, ten pieces of sensor data should be received. These ten pieces of sensor data may then be analyzed, combined with other data generated in monitoring process 40, and compared with the relevant REMS to inform the use of ingestible product 10 in patient 18.

Monitoring process 40 may be configured to generate 102 drug portion information 16 and providing 104 drug portion information 16 to third party 44, wherein drug portion information 16 may be based, at least in part, upon sensor data 12. Continuing with the above-stated example, drug portion information 16 may be configured as a dosage timeline as follows:

| Drug | Dosage | Date/Time |
|---|---|---|
| Rofecoxib | 25 milligrams | 1 Jan. 2018 @ 8:01 am |
| Rofecoxib | 25 milligrams | 2 Jan. 2018 @ 7:58 am |
| Rofecoxib | 25 milligrams | 3 Jan. 2018 @ 7:57 am |
| Rofecoxib | 25 milligrams | 3 Jan. 2018 @ 7:52 pm |
| Rofecoxib | 25 milligrams | 4 Jan. 2018 @ 8:00 am |
| Rofecoxib | 25 milligrams | 4 Jan. 2018 @ 8:15 pm |
| Rofecoxib | 25 milligrams | 10 Jan. 2018 @ 7:45 am |
| Rofecoxib | 25 milligrams | 11 Jan. 2018 @ 7:51 am |
| Rofecoxib | 25 milligrams | 12 Jan. 2018 @ 8:12 am |
| Rofecoxib | 25 milligrams | 13 Jan. 2018 @ 8:01 am |

Examples of third party 44 may include but are not limited to: a patient; a user; a medical professional; a regulatory authority; a pharmaceutical professional; and an insurance professional.

Patient: An example in which third party 44 is a patient is when patient 18 is provided with e.g., the above-illustrated dosage timeline that memorizes that patient 18 was administered or self-administered the above-described prescription (i.e., ten pills over a course of thirteen days).

User: An example in which third party 44 is a user is when e.g., a caretaker/relative of patient 18 is provided with e.g., the above-illustrated dosage timeline that memorizes that patient 18 was administered or self-administered the above-described prescription (i.e., ten pills over a course of thirteen days).

Medical Professional: An example in which third party 44 is a medical professional is when a medical professional (e.g., the prescribing physician that is treating patient 18) is provided with e.g., the above-illustrated dosage timeline that memorizes that patient 18 was administered or self-administered the above-described prescription (i.e., ten pills over a course of thirteen days).

Regulatory Authority: An example in which third party 44 is a regulatory authority is when a regulatory authority (e.g. FDA) is provided with e.g., the above-illustrated dosage timeline that memorizes that patient 18 was administered or self-administered the above-described prescription (i.e., ten pills over a course of thirteen days)

Pharmaceutical Professional: An example in which third party 44 is a pharmaceutical professional is when e.g., the manufacturer of drug portion 22 (e.g., rofecoxib) is provided with e.g., the above-illustrated dosage timeline that memorizes that patient 18 was administered or self-administered the above-described prescription (i.e., ten pills over a course of thirteen days).

Insurance Professional: An example in which third party 44 is an insurance professional is when an insurance professional associated with patient 18 is provided with e.g., the above-illustrated dosage timeline that memorizes that patient 18 was administered or self-administered the above-described prescription (i.e., ten pills over a course of thirteen days).

Monitoring process 40 may further be configured to receive 106 supplemental data 46 from a user, examples of which may include but are not limited to patient 18 and/or a caretaker/relative of patient 18. When such supplemental data 46 is received 106 from the user, drug portion information 16 may be based, at least in part, upon supplemental data 106. Examples of supplemental data 46 may include but is not limited to: reaction information; condition information; status information; and biographical information.

Reaction Information: An example of such reaction information may include but is not limited e.g., patient 18 (or a caregiver/relative of the same) providing supplemental data 46 that defines any adverse reactions (e.g., lightheadedness, fever, nausea, edema, palpitations) that patient 18 is experiencing concurrently or in association to drug portion 20.

Condition Information: An example of such condition information may include but is not limited e.g., patient 18 (or a caregiver/relative of the same) providing supplemental data 46 that defines the medical condition (e.g., body temperature, pulse rate. blood pressure, heart rhythm) of patient 18 while taking drug portion 20.

Status Information: An example of such status information may include but is not limited e.g., patient 18 (or a caregiver/relative of the same) providing supplemental data 46 that defines the general condition (e.g., tired, less aches, more soreness, ambulatory condition) of patient 18 while taking drug portion 20.

Biographical Information: An example of such biographical information may include but is not limited e.g., patient 18 (or a caregiver/relative of the same) providing supplemental data 46 that defines biographical information (e.g., name, age, weight, gender) for patient 18.

The above described supplemental information 46 may be proactively provided by the user or reactively entered by the user. For example, monitoring process 40 may be configured to prompt the user to provide supplemental information 46. For example, monitoring process 40 may ask the user e.g., “How are you feeling?”; “Are you experiencing any shortness of breath?”; “How was your knee pain this morning?”; “You appear to be experiencing a known side effect of amisulpride, please call your physician”; and/or “You exceeded the total daily recommended dose of rofecoxib today, please call your physician”.

### General:

As will be appreciated by one skilled in the art, the present disclosure may be embodied as a method, a system, or a computer program product. Accordingly, the present disclosure may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, the present disclosure may take the form of a computer program product on a computer-usable storage medium having computer-usable program code embodied in the medium.

Any suitable computer usable or computer readable medium may be utilized. The computer-usable or computer-readable medium may be, for example but not limited to, an electronic, magnetic, optical, electromagnetic, infrared, or semiconductor system, apparatus, device, or propagation medium. More specific examples (a non-exhaustive list) of the computer-readable medium may include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a transmission media such as those supporting the Internet or an intranet, or a magnetic storage device. The computer-usable or computer-readable medium may also be paper or another suitable medium upon which the program is printed, as the program can be electronically captured, via, for instance, optical scanning of the paper or other medium, then compiled, interpreted, or otherwise processed in a suitable manner, if necessary, and then stored in a computer memory. In the context of this document, a computer-usable or computer-readable medium may be any medium that can contain, store, communicate, propagate, or transport the program for use by or in connection with the instruction execution system, apparatus, or device. The computer-usable medium may include a propagated data signal with the computer-usable program code embodied therewith, either in baseband or as part of a carrier wave. The computer usable program code may be transmitted using any appropriate medium, including but not limited to the Internet, wireline, optical fiber cable, RF, etc.

Computer program code for carrying out operations of the present disclosure may be written in an object oriented programming language such as Java, Smalltalk, C++ or the like. However, the computer program code for carrying out operations of the present disclosure may also be written in conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through a local area network/a wide area network/the Internet (e.g., network 14).

The present disclosure is described with reference to flowchart illustrations and/or block diagrams of methods, apparatus (systems) and computer program products according to embodiments of the disclosure. It will be understood that each block of the flowchart illustrations and/or block diagrams, and combinations of blocks in the flowchart illustrations and/or block diagrams, may be implemented by computer program instructions. These computer program instructions may be provided to a processor of a general purpose computer/special purpose computer/other programmable data processing apparatus, such that the instructions, which execute via the processor of the computer or other programmable data processing apparatus, create means for implementing the functions/acts specified in the flowchart and/or block diagram block or blocks.

These computer program instructions may also be stored in a computer-readable memory that may direct a computer or other programmable data processing apparatus to function in a particular manner, such that the instructions stored in the computer-readable memory produce an article of manufacture including instruction means which implement the function/act specified in the flowchart and/or block diagram block or blocks.

The computer program instructions may also be loaded onto a computer or other programmable data processing apparatus to cause a series of operational steps to be performed on the computer or other programmable apparatus to produce a computer implemented process such that the instructions which execute on the computer or other programmable apparatus provide steps for implementing the functions/acts specified in the flowchart and/or block diagram block or blocks.

The flowcharts and block diagrams in the figures may illustrate the architecture, functionality, and operation of possible implementations of systems, methods and computer program products according to various embodiments of the present disclosure. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustrations, and combinations of blocks in the block diagrams and/or flowchart illustrations, may be implemented by special purpose hardware-based systems that perform the specified functions or acts, or combinations of special purpose hardware and computer instructions.

The terminology used herein is for the purpose of describing particular embodiments only and is not intended to be limiting of the disclosure. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements in the claims below are intended to include any structure, material, or act for performing the function in combination with other claimed elements as specifically claimed. The description of the present disclosure has been presented for purposes of illustration and description, but is not intended to be exhaustive or limited to the disclosure in the form disclosed. Many modifications and variations will be apparent to those of ordinary skill in the art without departing from the scope and spirit of the disclosure. The embodiment was chosen and described in order to best explain the principles of the disclosure and the practical application, and to enable others of ordinary skill in the art to understand the disclosure for various embodiments with various modifications as are suited to the particular use contemplated.

A number of implementations have been described. Having thus described the disclosure of the present application in detail and by reference to embodiments thereof, it will be apparent that modifications and variations are possible without departing from the scope of the disclosure defined in the appended claims.

What is claimed is:

1. An ingestible product, configured to be swallowed by a patient, comprising:
a drug portion that requires a Risk Evaluation and Mitigation Strategy (REMS) plan; and
a wireless sensor portion configured to transmit sensor data concerning the drug portion to a remote device.

2. The ingestible product of claim 1 wherein the drug portion is chosen from the group consisting of:
a COX-2 Selective Nonsteroidal Anti-inflammatory Drug (NSAID); and
a D2 receptor antagonist.

3. The ingestible product of claim 1 wherein the wireless sensor portion in configured to wirelessly transmit the sensor data via a wireless communication protocol.

4. The ingestible product of claim 3 wherein the wireless communication protocol is configured to couple the wireless sensor portion to the remote device.

5. The ingestible product of claim 3 wherein the sensor data includes:
identification information concerning the drug portion; and
dosage information concerning the drug portion.

6. The ingestible product of claim 3 wherein the remote device is chosen from the group consisting of:
a smart phone;
a smart watch;
a wearable patch;
a dedicated external device; and
a remote computing system.

7. A computer-implemented method, executed on a computing device, comprising:
receiving sensor data from a wireless sensor portion of an ingestible product, wherein the sensor data concerns a drug portion of the ingestible product that requires a Risk Evaluation and Mitigation Strategy (REMS) plan;
generating drug portion information; and
providing the drug portion information to a third party;
wherein the drug portion information is based, at least in part, upon the sensor data.

8. The computer-implemented method of claim 7 further comprising:
receiving supplemental data from a user;
wherein the drug portion information is based, at least in part, upon the supplemental data.

9. The computer-implemented method of claim 8 wherein the supplemental data is chosen from the group consisting of:
reaction information;
condition information;
status information; and
biographical information.

10. The computer-implemented method of claim 7 wherein the ingestible product is chosen from the group consisting of a:
a pill;
a capsule; and
a gelcap.

11. The computer-implemented method of claim 7 wherein the drug portion is chosen from the group consisting of:
a COX-2 Selective Nonsteroidal Anti-inflammatory Drug (NSAID); and
a D2 receptor antagonist.

12. The computer-implemented method of claim 7 wherein the third party is chosen from the group consisting of:
a patient;
a user;
a medical professional;
a regulatory authority;
a pharmaceutical professional; and
an insurance professional.

13. The computer-implemented method of claim 7 wherein the sensor data includes:
identification information concerning the drug portion; and
dosage information concerning the drug portion.

14. A computer program product residing on a computer readable medium having a plurality of instructions stored thereon which, when executed by a processor, cause the processor to perform operations comprising:
receiving sensor data from a wireless sensor portion of an ingestible product, wherein the sensor data concerns a drug portion of the ingestible product that requires a Risk Evaluation and Mitigation Strategy (REMS) plan;
generating drug portion information; and
providing the drug portion information to a third party;
wherein the drug portion information is based, at least in part, upon the sensor data.

15. The computer program product of claim 14 further comprising:
receiving supplemental data from a user;
wherein the drug portion information is based, at least in part, upon the supplemental data.

16. The computer program product of claim 15 wherein the supplemental data is chosen from the group consisting of:
reaction information;
condition information;
status information; and
biographical information.

17. The computer program product of claim 14 wherein the ingestible product is chosen from the group consisting of a:
a pill;
a capsule; and
a gelcap.

18. The computer program product of claim 14 wherein the drug portion is chosen from the group consisting of:
a COX-2 Selective Nonsteroidal Anti-inflammatory Drug (NSAID); and
a D2 receptor antagonist.

19. The computer program product of claim 14 wherein the third party is chosen from the group consisting of:
a patient;
a user;
a medical professional;
a regulatory authority;
a pharmaceutical professional; and
an insurance professional.

20. The computer program product of claim 14 wherein the sensor data includes:
identification information concerning the drug portion; and
dosage information concerning the drug portion.
